(19) United States
(12) Patent Application Publication
(10) Pub. No.: US 2024/0081707 A1
(43) Pub. Date: Mar. 14, 2024

(54) STRESS LEVEL ESTIMATION METHOD, TRAINING DATA GENERATION METHOD, AND STORAGE MEDIUM

(71) Applicant: NEC Corporation, Minato-ku, Tokyo (JP)

(72) Inventor: Yoshiki NAKASHIMA, Tokyo (JP)

(73) Assignee: NEC Corporation, Minato-ku, Tokyo (JP)

(21) Appl. No.: 18/272,285

(22) PCT Filed: Jan. 18, 2021
(86) PCT No.: PCT/JP2021/001494

§ 371 Date: Jul. 13, 2023

(51) Int. Cl. A61B 5/16; A61B 5/00; G16H 50/70

(52) U.S. Cl. CPC A61B 5/165; A61B 5/7267; G16H 50/70; A61B 5/681; G16H 10/20

(57) ABSTRACT

In order to estimate a stress level with higher accuracy than conventional techniques, a stress level estimation method includes: classifying, by at least one processor, measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and estimating, by the at least one processor, a stress level of the subject using at least one of the first measurement data and the second measurement data.

### TECHNICAL FIELD

The present invention relates to a method and the like for estimating a stress level of a subject using measurement data.

### BACKGROUND ART

In recent years, there are increasing cases where employees suffer from mental problems such as depression due to occupational stress, resulting in quitting jobs or taking leaves of absence. Along with the circumstances, there is also a problem of increasing burdens on companies that maintain and secure employees. Against this background, studies on stress monitoring are underway. For example, studies are also underway on a technique for estimating a stress level of a subject using measurement data such as body motion data and biological data of the subject.

### CITATION LIST

### Non-Patent Literature

[Non-patent Literature 1]
Yuko Kai, et. al., “ASSOCIATION OF LEISURE TIME PHYSICAL ACTIVITY ON DEPRESSIVE SYMPTOMS WITH JOB STRAIN”, Bulletin of the Physical Fitness Research Institute, No. 107, pp. 1-10, April, 2009
[Non-patent Literature 2]
Ryoichi Inaba, et. al., “Relationship of Burnout or Job Stress with Physical Activity among Female Hospital Nurses”, Japanese Journal of Occupational Medicine and Traumatology, 66, pp. 253-258, 2018

### SUMMARY OF INVENTION

### Technical Problem

As will be described below, there is room for improving estimation accuracy in conventional stress level estimation as described above. For example, it is highly possible that body motion data measured while the subject is off work is derived from physical activity based on free will of the subject, such as leisure and sport. Here, according to Non-Patent Literature 1, it is indicated that a ratio of having a depressed state is lower in persons who perform physical activities in leisure time, as compared with persons who do not. Therefore, it is considered that body motion data measured while a subject is off work has a negative correlation with a stress level.

Meanwhile, it is highly possible that body motion data measured while the subject is working is derived from job-related physical activity. Here, Non-Patent Literature 2 suggests, as one of the burnout factors of a female hospital nurse, an increase in physical activity during work due to increased occupational stress. Therefore, it is considered that body motion data measured while a subject is working has a positive correlation with a stress level.

As such, whether a value of measurement data contributes to an increase or a decrease in stress level may vary depending on whether the subject is within working hours or outside the working hours when the measurement data is measured. Conventionally, the stress level has been estimated without taking this point into consideration. Therefore, there was room for improvement in estimation accuracy.

An example object of an example aspect of the present invention is to provide a stress level estimation method and the like that make it possible to estimate a stress level with higher accuracy than conventional techniques.

### Solution to Problem

A method for estimating a stress level according to an example aspect of the present invention includes: classifying, by at least one processor, measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and estimating, by the at least one processor, a stress level of the subject using at least one of the first measurement data and the second measurement data.

A method for generating training data according to an example aspect of the present invention includes: classifying, by at least one processor, measurement data into first measurement data and second measurement data, the measurement data pertaining to a stress level that indicates a degree of stress of each of one or more subjects, the first measurement data having been measured during working hours of the subject, and the second measurement data having been measured outside the working hours; and generating, by the at least one processor, at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

An information processing apparatus according to an example aspect of the present invention includes: a classification means that classifies measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and an estimation means that estimates a stress level of the subject using at least one of the first measurement data and the second measurement data.

An information processing apparatus according to an example aspect of the present invention includes: a classification means that classifies measurement data into first measurement data and second measurement data, the measurement data pertaining to a stress level that indicates a degree of stress of each of one or more subjects, the first measurement data having been measured during working hours of the subject, and the second measurement data having been measured outside the working hours; and a training data generation means that generates at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

A stress level estimation program according to an example aspect of the present invention is a program for causing a computer to function as an information processing apparatus, the stress level estimation program causing the computer to function as: a classification means that classifies measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and an estimation means that estimates a stress level of the subject using at least one of the first measurement data and the second measurement data.

A training data generation program according to an example aspect of the present invention is a program for causing a computer to function as an information processing apparatus, the training data generation program causing the computer to function as: a classification means that classifies measurement data into first measurement data and second measurement data, the measurement data pertaining to a stress level that indicates a degree of stress of each of one or more subjects, the first measurement data having been measured during working hours of the subject, and the second measurement data having been measured outside the working hours; and a training data generation means that generates at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

### Advantageous Effects of Invention

According to an example aspect of the present invention, it is possible to estimate a stress level with higher accuracy.

### BRIEF DESCRIPTION OF DRAWINGS

FIG. 1 is a flowchart illustrating flows of a training data generation method, an estimation model generation method, and a stress level estimation method according to a first example embodiment of the present invention.

FIG. 2 is a block diagram illustrating a configuration of an information processing apparatus according to the first example embodiment of the present invention.

FIG. 3 is a diagram illustrating an overview of a process carried out by an information processing apparatus according to a second example embodiment of the present invention.

FIG. 4 is a block diagram illustrating a configuration of the information processing apparatus.

FIG. 5 is a diagram illustrating an example of a triaxial acceleration histogram.

FIG. 6 is a flowchart illustrating a flow of a training data generation method according to the second example embodiment of the present invention.

FIG. 7 is a flowchart illustrating a flow of a stress level estimation method according to the second example embodiment of the present invention.

FIG. 8 is a diagram illustrating overviews of a training data generation method, an estimation model generation method, and a stress level estimation method according to a third example embodiment of the present invention.

FIG. 9 is a diagram illustrating an example of a computer which executes instructions of a program that is software realizing functions of the information processing apparatus.

### EXAMPLE EMBODIMENTS

### First Example Embodiment

The following description will discuss a first example embodiment of the present invention in detail with reference to the drawings. The present example embodiment is a basic form of example embodiments described later.

(Flows of Training Data Generation Method, Estimation Model Generation Method, and Stress Level Estimation Method)

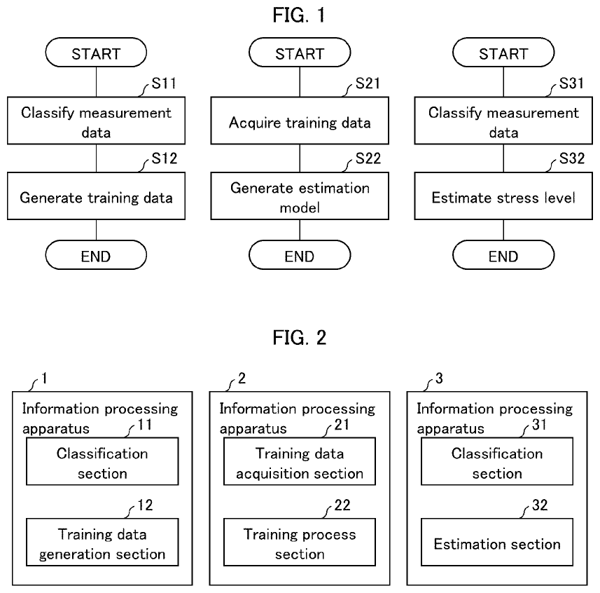

FIG. 1 is a flowchart illustrating flows of a training data generation method, an estimation model generation method, and a stress level estimation method according to the first example embodiment of the present invention. Note that S11 and S12 indicate the training data generation method, S21 and S22 indicate the estimation model generation method, and S31 and S32 indicate the stress level estimation method.

(Training Data Generation Method)

In S11, at least one processor classifies measurement data into first measurement data and second measurement data. Here, the measurement data pertains to a stress level that indicates a degrees of stress of each of one or more subjects. The first measurement data is data which has been measured during working hours of the subject. The second measurement data is data which has been measured outside the working hours.

In S12, the at least one processor generates at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity. Thus, the training data generation method ends.

The processes of S11 and S12 may be repeated until a necessary number of pieces of training data are generated. Pieces of measurement data in respective repetitions can be pieces of measurement data measured for the same subject or can be pieces of measurement data measured for different subjects. Note, however, that when pieces of measurement data of different subjects are used, it is preferable, from the viewpoint of increasing accuracy in estimating a stress level, to use pieces of measurement data of subjects whose attributes (such as age, gender, and occupation) are as close as possible.

Note that the stress level in the first training data indicates a degree of stress which a subject suffers when the first measurement data has been measured. Similarly, the stress level in the second training data indicates a degree of stress which a subject suffers when the second measurement data has been measured. The stress level in the third training data indicates a degree of stress of a subject in a measurement period of all pieces of measurement data including the first measurement data and the second measurement data.

As described above, the training data generation method according to the present example embodiment includes: classifying, by the at least one processor, measurement data pertaining to a stress level that indicates a degree of stress of each of one or more subjects into first measurement data and second measurement data; and generating, by the at least one processor, at least one of first training data, second training data, and third training data.

Therefore, by using training data generated by the training data generation method according to the present example embodiment, it is possible to bring about an example advantage of constructing an estimation model that is capable of estimating a stress level while taking into consideration whether a subject is within working hours or outside the working hours when measurement data is measured. Note that the processes of S11 and S12 may be carried out by a single processor, or the processes of S11 and S12 may be carried out by separate processors. In the latter case, the processors can be provided in a single information processing apparatus or can be provided in respective different information processing apparatuses. This also applies to S21 and S22 and S31 through S33, which will be described below.

(Estimation Model Generation Method)

In S21, at least one processor acquires at least one of first training data, second training data, and third training data. Note that the first training data is training data in which a stress level of a subject is associated with a first feature quantity calculated from first measurement data. The second training data is training data in which a stress level of a subject is associated with a second feature quantity calculated from second measurement data. The third training data is training data in which a stress level of a subject is associated with the first feature quantity and the second feature quantity.

In S22, the at least one processor generates at least one of (1) a first estimation model for which the first feature quantity is used as an explanatory variable, the first estimation model being generated by training using the first training data, (2) a second estimation model for which the second feature quantity is used as an explanatory variable, the second estimation model being generated by training using the first training data, and (3) a third estimation model for which the first feature quantity and the second feature quantity are used as explanatory variables, the third estimation model being generated by training using the third training data. Thus, the estimation model generation method ends.

As described above, the estimation model generation method according to the present example embodiment includes: acquiring, by the at least one processor, at least one of the first training data, the second training data, and the third training data which have been generated by the foregoing training data generation method; and, by the at least one processor, at least one of generating a first estimation model, generating a second estimation model, and generating a third estimation model.

Therefore, according to the estimation model generation method of the present example embodiment, it is possible to bring about an example advantage of constructing an estimation model that is capable of estimating a stress level while taking into consideration whether a subject is within working hours or outside the working hours when measurement data is measured. Note that estimation algorithms of the foregoing estimation models are not particularly limited. The estimation algorithm can be, for example, a nonlinear model such as a neural network model or a linear model such as linear regression.

(Stress Level Estimation Method)

In S31, at least one processor classifies measurement data into first measurement data and second measurement data. Here, the measurement data is data which has been measured during a predetermined time period, and pertains to a stress level that indicates a degrees of stress of a subject. The first measurement data is data which has been measured during working hours of the subject. The second measurement data is data which has been measured outside the working hours. This subject is a subject whose stress level is to be estimated.

The “subject” of the measurement data in S31 can be the same person as the “subject” in S11 described above, that is, the subject from whom measurement data used to generate training data has been measured, or can be a different person. Note, however, that, from the viewpoint of increasing accuracy in estimating a stress level, it is preferable that the “subject” of measurement data in S31 is the person from whom measurement data used to generate training data has been measured or a person whose attributes (such as age, gender, and occupation) are as close as possible to that person.

In S32, the at least one processor estimates the stress level of the subject using at least one of the first measurement data and the second measurement data. Thus, the stress level estimation method ends.

As described above, the stress level estimation method according to the present example embodiment includes: classifying, by the at least one processor, measurement data measured during a predetermined time period into first measurement data and second measurement data; and estimating, by the at least one processor, the stress level of the subject using at least one of the first measurement data and the second measurement data.

Therefore, according to the stress level estimation method of the present example embodiment, it is possible to bring about an example advantage of estimating a stress level with higher accuracy while taking into consideration whether a subject is within working hours or outside the working hours when measurement data is measured.

(Configurations of Information Processing Apparatuses 1 Through 3)

The following description will discuss a configuration of each of information processing apparatuses 1 through 3 according to the present example embodiment, with reference to FIG. 2. FIG. 2 is a block diagram illustrating the configuration of each of the information processing apparatuses 1 through 3. The information processing apparatus 1 is an apparatus that generates training data for constructing a stress level estimation model. The information processing apparatus 2 is an apparatus that constructs a stress level estimation model. The information processing apparatus 3 is an apparatus that estimates a stress level of a subject.

(Configuration of Information Processing Apparatus 1)

The information processing apparatus 1 includes a classification section 11 and a training data generation section 12. The classification section 11 classifies measurement data pertaining to a stress level of each of one or more subjects into first measurement data and second measurement data. This process corresponds to S11 in FIG. 1. Then, the training data generation section 12 generates at least one of (1) through (3) below. This process corresponds to S12 in FIG. 1.
(1) First training data in which a stress level of the subject is associated with a first feature quantity calculated from the first measurement data.
(2) Second training data in which a stress level of the subject is associated with a second feature quantity calculated from the second measurement data.
(3) Third training data in which a stress level of the subject is associated with the first feature quantity and the second feature quantity.

As described above, the information processing apparatus 1 according to the present example embodiment employs the configuration of including: the classification section 11 that classifies measurement data into first measurement data and second measurement data; and the training data generation section 12 that generates at least one of first training data, second training data, and third training data. Therefore, by using training data generated by the information processing apparatus 1 according to the present example embodiment, it is possible to bring about an example advantage of constructing an estimation model that is capable of estimating a stress level while taking into consideration whether a subject is within working hours or outside the working hours when measurement data is measured.

Note that the functions of the information processing apparatus 1 described above can also be realized by a program. A training data generation program according to the present example embodiment is a program for causing a computer to function as an information processing apparatus, the program causing the computer to function as: the classification section 11 that classifies measurement data pertaining to a stress level of each of one or more subjects into first measurement data and second measurement data; and the training data generation section 12 that generates at least one of first training data, second training data, and third training data. As such, according to the training data generation program of the present example embodiment, training data is generated after carrying out classification based on whether a subject is within working hours or outside the working hours when measurement data is measured. Therefore, by training using the training data, it is possible to construct an estimation model that is capable of estimating a stress level while taking into consideration whether a subject is within working hours or outside the working hours when measurement data is measured.

(Configuration of Information Processing Apparatus 2)

The information processing apparatus 2 includes a training data acquisition section 21 and a training process section 22. The training data acquisition section 21 acquires at least one of first training data, second training data, and third training data. This process corresponds to S21 in FIG. 1. The training process section 22 generates at least one of a first estimation model, a second estimation model, and a third estimation model. This process corresponds to S22 in FIG. 1. According to this configuration, it is possible to construct an estimation model that is capable of estimating a stress level while taking into consideration whether a subject is within working hours or outside the working hours when measurement data is measured.

(Configuration of Information Processing Apparatus 3)

The information processing apparatus 3 includes a classification section 31 and an estimation section 32. The classification section 31 classifies measurement data into first measurement data and second measurement data. This process corresponds to S31 in FIG. 1. The estimation section 32 estimates a stress level of a subject using at least one of the first measurement data and the second measurement data. This process corresponds to S32 in FIG. 1.

As described above, the information processing apparatus 3 according to the present example embodiment employs the configuration of including: the classification section 31 that classifies measurement data measured during a predetermined time period into first measurement data and second measurement data; and the estimation section 32 that estimates a stress level of a subject using at least one of the first measurement data and the second measurement data. Therefore, according to the information processing apparatus 3 of the present example embodiment, it is possible to bring about an example advantage of estimating a stress level with higher accuracy while taking into consideration whether a subject is within working hours or outside the working hours when measurement data is measured.

The functions of the information processing apparatus 3 described above can also be realized by a program. That is, a stress level estimation program according to the present example embodiment is a program for causing a computer to function as the information processing apparatus 3, the program causing the computer to function as: the classification section 31 that classifies measurement data measured during a predetermined time period into first measurement data and second measurement data; and the estimation section 32 that estimates a stress level of a subject using at least one of the first measurement data and the second measurement data. Therefore, according to the stress level estimation program of the present example embodiment, it is possible to estimate a stress level with higher accuracy while taking into consideration whether a subject is within working hours or outside the working hours when measurement data is measured.

### Second Example Embodiment

The following description will discuss a second example embodiment of the present invention in detail with reference to the drawings. The same reference numerals are given to constituent elements which have functions identical with those described in the first example embodiment, and descriptions as to such constituent elements are omitted as appropriate. This also applies to a third example embodiment described later.

(Overview)

In the present example embodiment, an example will be described in which a single information processing apparatus carries out: generation of training data for constructing a stress level estimation model; construction of an estimation model using the training data; and estimation of a stress level using the estimation model. The information processing apparatus is referred to as an information processing apparatus 4.

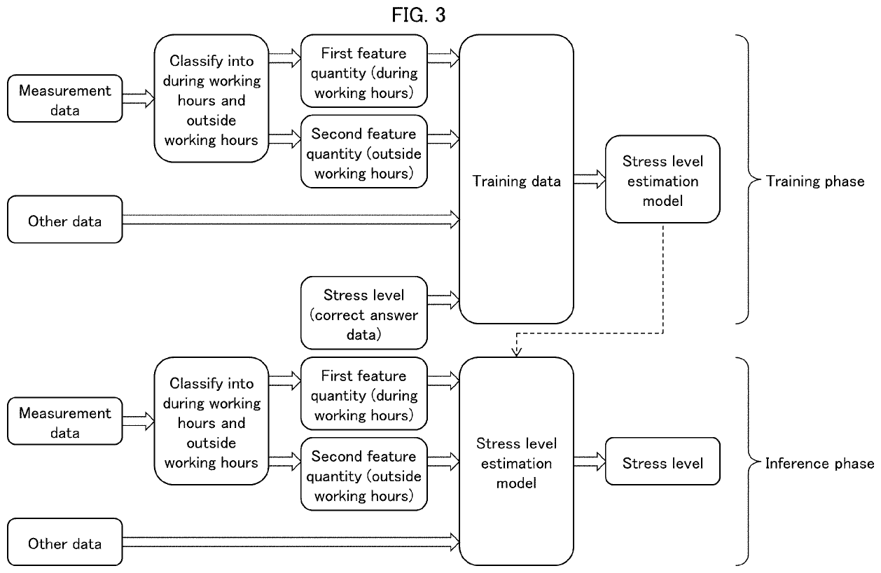

FIG. 3 is a diagram illustrating an overview of a process carried out by the information processing apparatus 4. In a training phase, the information processing apparatus 4 generates training data using (i) measurement data which has been measured during a predetermined time period and pertains to a stress level that indicates a degree of stress of a subject, and (ii) other pieces of data that are correlated with the degree of stress of the subject. Examples of those other pieces of data include data indicating a body temperature and the like of a subject, as well as biological signal data such as perspiration, brain waves, pulse, and heart rate.

The information processing apparatus 4 classifies, among pieces of data used to generate training data, measurement data into first measurement data measured during working hours of the subject and second measurement data measured outside the working hours. Then, the information processing apparatus 4 calculates a first feature quantity from the first measurement data, and calculates a second feature quantity from the second measurement data. Note that feature quantities may be calculated from other pieces of data. The calculation of a feature quantity can also be referred to as extraction of a feature quantity.

Then, the information processing apparatus 4 generates training data while associating a combination of the first feature quantity, the second feature quantity, and the other pieces of data with a stress level, as correct answer data, of the subject during the time period in which the measurement data has been measured. The information processing apparatus 4 carries out machine learning using a plurality of pieces of training data thus generated to generate a stress level estimation model.

In an inference phase, the information processing apparatus 4 estimates a stress level of a subject using the estimation model which has been generated in the training phase. Specifically, first, the information processing apparatus 4 acquires (i) measurement data which has been measured during a predetermined time period and pertains to a stress level that indicates a degrees of stress of a subject and (ii) other pieces of data that are correlated with the degree of stress of the subject. Next, the information processing apparatus 4 classifies the acquired measurement data into first measurement data measured during working hours of the subject and second measurement data measured outside the working hours. Then, the information processing apparatus 4 calculates a first feature quantity from the first measurement data, calculates a second feature quantity from the second measurement data, and inputs the calculated feature quantities and the acquired other pieces of data into the stress level estimation model. Thus, an estimation value of the stress level of the subject can be obtained.

(Configuration of Information Processing Apparatus 4)

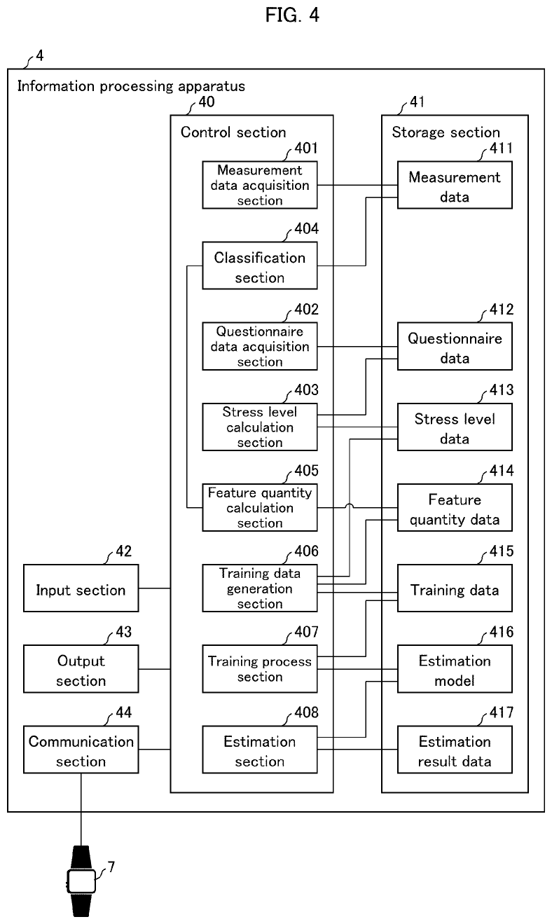

The following description will discuss a configuration of the information processing apparatus 4 with reference to FIG. 4. FIG. 4 is a block diagram illustrating the configuration of the information processing apparatus 4. FIG. 4 also illustrates a wearable terminal 7 as an example of an apparatus that measures measurement data.

The wearable terminal 7 includes a triaxial acceleration sensor and transmits an output value of the acceleration sensor as measurement data to the information processing apparatus 4. When the subject wears the wearable terminal 7, body motion of the subject is detected by the acceleration sensor. Since it has been found that body motion is correlated with the stress level of the subject, it is possible to estimate a stress level by using an output value of the acceleration sensor as measurement data. Note that the acceleration sensor is not limited to a triaxial acceleration sensor, and may be a uniaxial or biaxial acceleration sensor.

The information processing apparatus 4 includes a control section 40 that comprehensively controls components of the information processing apparatus 4, and a storage section 41 that stores various kinds of data used by the information processing apparatus 4. The information processing apparatus 4 further includes: an input section 42 that receives input of data with respect to the information processing apparatus 4; an output section 43 for outputting data from the information processing apparatus 4; and a communication section 44 for carrying out communication between the information processing apparatus 4 and another apparatus (e.g., the wearable terminal 7).

The control section 40 includes a measurement data acquisition section 401, a questionnaire data acquisition section 402, a stress level calculation section 403, a classification section 404, a feature quantity calculation section 405, a training data generation section 406, a training process section 407, and an estimation section 408. The storage section 41 stores measurement data 411, questionnaire data 412, stress level data 413, feature quantity data 414, training data 415, an estimation model 416, and estimation result data 417.

The measurement data acquisition section 401 acquires measurement data pertaining to the stress level of the subject and causes the storage section 41 to store the acquired measurement data. The measurement data stored in the storage section 41 is measurement data 411. The measurement data 411 can include data used for generation of training data 415 and data used for estimation of a stress level.

The questionnaire data acquisition section 402 acquires a result of a questionnaire pertaining to the stress level of the subject in a time period in which measurement data 411 (that is used for generation of training data 415) has been measured, and causes the storage section 41 to store questionnaire data 412 indicating the acquired result. This questionnaire is a questionnaire answered by the subject in order to calculate the stress level of the subject. The questionnaire only needs to have content that reflects a stress level of a subject, and may be a stress questionnaire of, for example, perceived stress scale (PSS). The stress questionnaire of PSS is a questionnaire in the form in which a subject selects an applicable one from a plurality of options, for each of a plurality of questions regarding how the subject feels and behaves during a time period in question.

The stress level calculation section 403 calculates a stress level of a subject using the questionnaire data 412, and causes the storage section 41 to store stress level data 413 that indicates the calculated stress level. Any method for calculating the stress level can be applied. For example, in a case where the questionnaire data 412 is data indicating a result of a stress questionnaire of PSS, the stress level calculation section 403 calculates a PSS score.

The classification section 404 classifies the measurement data 411 into first measurement data measured during working hours of the subject and second measurement data measured outside the working hours. A classification method by the classification section 404 is not particularly limited, as long as the measurement data 411 can be classified into first measurement data and second measurement data. Specific examples of the classification method carried out by the classification section 404 will be described later.

The feature quantity calculation section 405 calculates a first feature quantity from the first measurement data, calculates a second feature quantity from the second measurement data, and causes the storage section 41 to store the calculated first and second feature quantities. Data which indicates the first and second feature quantities and which the feature quantity calculation section 405 has caused the storage section 41 to store is feature quantity data 414.

The training data generation section 406 generates training data while associating, as correct answer data, the stress level indicated by the stress level data 413 with the first feature quantity and the second feature quantity indicated by the feature quantity data 414. This training data corresponds to the third training data in the above described first example embodiment. Then, the training data generation section 406 causes the storage section 41 to store the generated training data as training data 415.

The training process section 407 generates, by training using the training data 415, an estimation model for which the first feature quantity and the second feature quantity are used as explanatory variables and from which a stress level is obtained as an objective variable. This estimation model corresponds to the third estimation model in the above described first example embodiment. Then, the training process section 407 causes the storage section 41 to store the generated estimation model as an estimation model 416.

The estimation section 408 estimates a stress level of a subject using the first measurement data and the second measurement data. More specifically, the estimation section 408 inputs, into the estimation model 416, feature quantity data 414 that indicates a first feature quantity calculated using the first measurement data and a second feature quantity calculated using the second measurement data, and thus calculates an estimation value of the stress level. Then the estimation section 408 causes the storage section 41 to store estimation result data 417 indicating a stress level estimation result.

(Example of Classification Method)

The following description will discuss an example of a method for classifying measurement data into first measurement data and second measurement data. For example, the classification section 404 can carry out classification using position information of the subject at a time when measurement data has been measured. In this case, position information of a place of work of the subject may be registered in advance. With the configuration, the classification section 404 can classify the measurement data into measurement data measured during working hours, that is, first measurement data, in a case where position information of the subject at the time when the measurement data has been measured indicates a position within a range that has been set based on the place of work. Then, the classification section 404 may classify measurement data that has not been classified as first measurement data into second measurement data. The position information of the subject may be acquired from, for example, a portable apparatus (having a global positioning system (GPS) function) such as the wearable terminal 7 or a smart phone carried by the subject.

For example, the classification section 404 may carry out classification based on an activity pattern of the subject. In this case, the classification section 404 may classify, into second measurement data, measurement data that has been measured when the activity pattern of the subject falls under an activity pattern (e.g., an activity pattern during sport) that is typical for a time period outside working hours. Similarly, the classification section 404 may classify, into first measurement data, measurement data that has been measured when the activity pattern of the subject falls under an activity pattern that is typical for a time period within working hours. Note that activity patterns typical for time periods outside working hours and within working hours may be registered in advance. The activity pattern of the subject may be identified by analyzing triaxial acceleration data measured by the wearable terminal 7 or the like.

In addition, for example, in a case where there is an activity pattern (e.g., moving on a train or a bicycle, or the like) typical for commuting, such an activity pattern may be registered in advance. In this case, the classification section 404 may detect a preregistered activity pattern and classify, into first measurement data, measurement data that is obtained during a predetermined time period (which may be appropriately determined based on general working hours) from the detection.

Of course, the method of classifying measurement data into first measurement data and second measurement data is not limited to the foregoing examples. For example, the classification section 404 may classify measurement data measured during general working hours (e.g., from 9 a.m. to 6 p.m. on weekdays) into first measurement data, and may classify measurement data measured during the other time zones into second measurement data. Note that, by registering working hours of a subject in advance, more accurate classification can be carried out based on the registered working hours.

(Example of Feature Quantity Calculation)

The following description will discuss an example of calculating a feature quantity in a case where measurement data 411 is triaxial acceleration data. Here, it is assumed that triaxial acceleration data is measured intermittently at constant sampling intervals of Tₛ (sec). Further, it is assumed that a serial number of acquired acceleration data is k (k=0 for acceleration data acquired first) and a maximum value of k is K (0 s k s K).

Assuming that x, y, and z components of triaxial acceleration data obtained at a time kTₛ are x(kTₛ), y(kTₛ), and z(kTₛ), respectively, a triaxial acceleration RMS(kTₛ) at the time is expressed by mathematical formula (1) below. The feature quantity calculation section 405 calculates RMS(kTₛ) for each of pieces of acceleration data from 0 to K included in the measurement data 411.

RMS(kTs)={square root over (x(kTs))²+(y(kTs))²+(z(kTs))²)} (1)

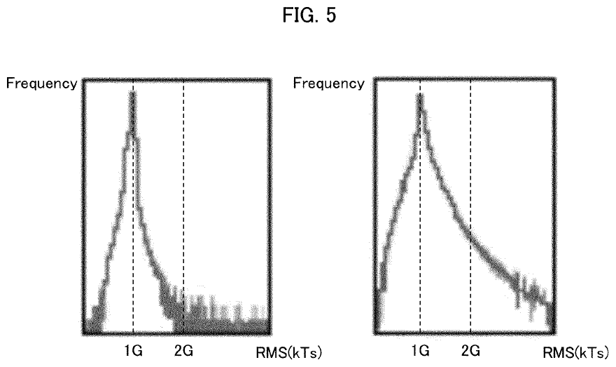

The RMS(kTₛ) calculated in this manner exhibits a feature of body motion of the subject. FIG. 5 is a diagram illustrating an example of a triaxial acceleration histogram. The horizontal axis of the histogram is the triaxial acceleration RMS(kTₛ), and the vertical axis is a frequency thereof. FIG. 5 shows two histograms. The left side is a histogram of triaxial acceleration in working hours on one day of a subject whose PSS-10 score is 11. Meanwhile, the right side is a histogram of triaxial acceleration in working hours on one day of a subject whose PSS-10 score is 26.

Note that the PSS-10 score is calculated based on a result of a predetermined questionnaire answered by a subject, and a higher value indicates higher stress. A PSS-10 score range is from 0 to 40. It can be said that a subject with a PSS-10 score of 11 is in a typical low stress state, and a subject with a PSS-10 score of 26 is in a typical high stress state.

Both of the two histograms illustrated in FIG. 5 are common in that the histograms have peaks around 1 G (G is gravitational acceleration), while there is a large difference in a range of 2 G or more. That is, in the histogram of the right side based on the acceleration data of the subject in the high stress state, the frequency in the range of 2 G or more is considerably higher than in the histogram of the left side based on the acceleration data of the subject in the low stress state. That is, it can be said that a large frequency of RMS(kTₛ) in the range of 2 G or more indicates that the stress level of the subject is high. In other words, it can be said that the frequency of RMS(kTₛ) in the range of 2 G or more is positively correlated with the stress level. This result is consistent with the view that body motion data measured when a subject is working has a positive correlation with the stress level, which is shown in Non-Patent Literature 2.

Therefore, the feature quantity calculation section 405 can calculate a feature quantity X(m) that is positively correlated with the stress level of the subject during a predetermined time period (e.g., one month) using measurement data (triaxial acceleration data in a time series) for the predetermined time period and using the following mathematical formulae (2) and (3).

$${RMS}_{m} ( {kT}_{s} ) = \{ \begin{matrix}1 & ( mw \leq RMS ( {kT}_{s} ) < ( m + 1 ) w ) \\ 0 & ( RMS ( {kT}_{s} ) < mw , ) ( m + 1 ) w \leq RMS ( {kT}_{s} ) )\end{matrix} \qquad ( 2 )$$

$$X ( m ) = \frac{\frac{1}{{KT}_{s}} {RMS}_{m} ( {kT}_{s} )}{\frac{1}{{KT}_{s}} {\sum }_{k = 0}^{K - 1} {RMS}_{m} ( {kT}_{s} )} \qquad ( 3 )$$

Mathematical formula (2) above is a formula for counting when RMS(kTₛ) is within a predetermined range. Specifically, RMSₘ(kTₛ) which is the left side of mathematical formula (2) above is 1 when RMS(kTₛ) is included in a range of mw or more and less than m(w+1), and is 0 when RMS(kTₛ) is not included in the range. Note that w is a width of the above range, and m is a coefficient. Moreover, a maximum value of m is M. M is set so that a maximum value of measurable triaxial acceleration is Mw.

X(m) shown in mathematical formula (3) above indicates a ratio of the RMSₘ(kTₛ) to a sum of the RMSₘ(kTₛ) for each of the pieces of acceleration data from 0 to K included in the measurement data 411.

A larger value of X(m) means that a frequency of RMS(kTₛ) within the above range (mw to m(w+1)) is relatively high. Therefore, according to X(m), it is possible to express a quantity of a relative frequency of RMS(kTₛ) in a predetermined range in accordance with a value of m that has been set.

For example, when w is 0.1 G and m is set to 20, the above range is 2 G to 2.1 G. The RMS(kTₛ) in this range is positively correlated with the stress level of the subject, as described with reference to FIG. 5. Therefore, X(m) determined based on w=0.1 G and m=20 can be used as a feature quantity that is positively correlated with the stress level.

Further, if the range of RMS(kTₛ) that is negatively correlated with the stress level is known, it is possible that X(m) in the range is obtained, and the obtained X(m) is used as a feature quantity that is negatively correlated with the stress level. For example, it has been found that RMS(kTₛ) in a range from 1 G to 2 G is negatively correlated with the stress level. Therefore, if w=0.1 G, X(m) can be obtained by setting m within a range of 10 to 20.

As described above, by using the acceleration data, it is possible to calculate a feature quantity that is positively correlated with the stress level and a feature quantity that is uncorrelated. The feature quantity calculation section 405 preferably calculates, among feature quantities that are correlated with the stress level as described above, a feature quantity that has a correlation reversed between a period during working hours and a period during working hours, that is, a feature quantity that has a positive correlation with the stress level during working hours and has a negative correlation with the stress level outside working hours. The feature quantity calculation section 405 may calculate a feature quantity that has a negative correlation with the stress level during working hours and has a positive correlation with the stress level outside working hours.

(Training Data Generation Method)

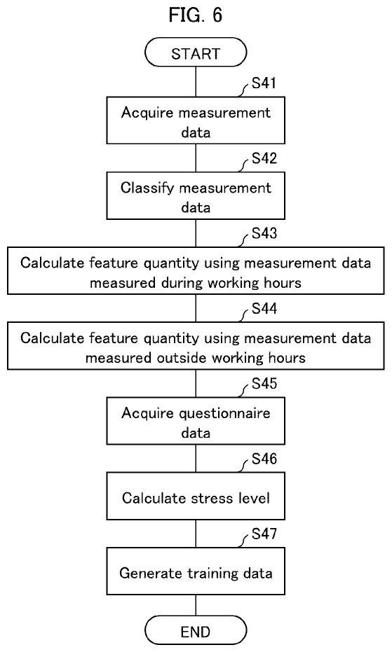

FIG. 6 is a flowchart illustrating a flow of a training data generation method according to the second example embodiment of the present invention. In the following descriptions, an example will be described in which training data is generated using, as measurement data, triaxial acceleration data of a subject measured by the wearable terminal 7. The measurement data to be used can be measurement data of a single subject or can be pieces of measurement data of a plurality of subjects. However, it is preferable that the measurement data used is measurement data of a subject whose response to stress is close to that of a subject whose stress level is to be estimated. In addition to the triaxial acceleration data, various kinds of biological data and the like can be used to generate training data. Further, in regard to each subject, it is assumed that a questionnaire has been answered for calculating a stress level during a period in which measurement data has been measured.

In S41, the measurement data acquisition section 41 acquires measurement data used for generation of training data. As described above, measurement data acquired here is triaxial acceleration data of a subject measured by the wearable terminal 7. Then, the measurement data acquisition section 41 causes the storage section 41 to store the acquired measurement data as measurement data 411.

In S42, the classification section 404 classifies the measurement data 411 into first measurement data measured during working hours of the subject and second measurement data measured outside the working hours. A classification result may be stored while associating the measurement data 411 with a label indicating the classification result. Since the classification method is as described above, descriptions thereof will not be repeated here.

In S43, the feature quantity calculation section 405 calculates a first feature quantity from the measurement data 411 which has been classified into the first measurement data in S42. In S44, the feature quantity calculation section 405 calculates a second feature quantity from the measurement data 411 which has been classified into the second measurement data in S42. These feature quantities are stored in the storage section 41 as feature quantity data 414. Note that the processes of S43 and S44 can be carried out concurrently or the process of S44 can be carried out first. Since the method for calculating the first feature quantity and the second feature quantity is as described above, descriptions thereof will not be repeated here.

In S45, the questionnaire data acquisition section 402 acquires questionnaire data indicating a result of the questionnaire for the subject in the measurement period of measurement data acquired in S41. Then, the questionnaire data acquisition section 402 causes the storage section 41 to store the acquired questionnaire data as questionnaire data 412.

In S46, the stress level calculation section 403 calculates a stress level of the subject using the questionnaire data 412. Then, the stress level calculation section 403 causes the storage section 41 to store the calculated stress level as stress level data 413. Note that the processes of S45 and S46 may be carried out before S41, or may be carried out concurrently with S41 through S44, as long as the processes of S45 and S46 are carried out before S47.

In S47, the training data generation section 406 generates training data while associating, as correct answer data, the stress level which has been calculated in S46 and which is indicated by the stress level data 413 with the first feature quantity and the second feature quantity which have been calculated in S43 and S44 and are indicated by the feature quantity data 414. Then, the training data generation section 406 causes the storage section 41 to store the generated training data as training data 415. Thus, the training data generation method ends.

(Stress Level Estimation Method)

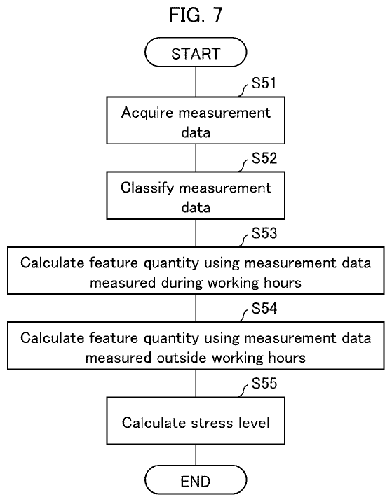

FIG. 7 is a flowchart illustrating a flow of a stress level estimation method according to the second example embodiment of the present invention. In the following descriptions, an example will be described in which a stress level of a subject in one month is estimated while using, as measurement data, triaxial acceleration data measured by the wearable terminal 7 for the one month. Note, however, that the measurement period can be less than one month or can be longer than one month.

In S51, the measurement data acquisition section 41 acquires measurement data. As described above, measurement data acquired here is triaxial acceleration data of a subject measured by the wearable terminal 7 for one month. Then, the measurement data acquisition section 41 causes the storage section 41 to store the acquired measurement data as measurement data 411.

In S52, the classification section 404 classifies the measurement data 411 into first measurement data measured during working hours of the subject and second measurement data measured outside the working hours. A classification result may be stored while associating the measurement data 411 with a label indicating the classification result.

In S53, the feature quantity calculation section 405 calculates a first feature quantity from the measurement data 411 which has been classified into the first measurement data in S52. In S54, the feature quantity calculation section 405 calculates a second feature quantity from the measurement data 411 which has been classified into the second measurement data in S52. The method for calculating the feature quantities is the same as that in S43 and S44 in FIG. 6. These feature quantities are stored in the storage section 41 as feature quantity data 414. Note that the processes of S53 and S54 can be carried out concurrently or the process of S54 can be carried out first.

In S55, the estimation section 408 estimates the stress level of the subject. Specifically, the estimation section 408 inputs, into the estimation model 416, the first feature quantity and the second feature quantity which have been calculated in S53 and S54 and are indicated in the feature quantity data 414. Note that, in a case where the estimation model 416 to be used includes data other than triaxial acceleration data (e.g., biological data and the like), the estimation section 408 also inputs such data into the estimation model 416. Then, the estimation section 408 causes the storage section 41 to store an output value of the estimation model 416 as estimation result data 417. Note that the estimation section 408 may cause the output section 43 to output the estimated stress level. Thus, the stress level estimation method ends.

As described above, the stress level estimation method according to the present example embodiment further includes: calculating, by the information processing apparatus 4, a first feature quantity from the first measurement data; and calculating, by the information processing apparatus 4, a second feature quantity from the second measurement data. The information processing apparatus 4 employs the configuration in which, in the estimating of the stress level, the stress level of the subject is estimated using the estimation model 416 for which the first feature quantity and the second feature quantity are used as explanatory variables, and from which a stress level is obtained as an objective variable. Therefore, according to the stress level estimation method of the present example embodiment, it is possible to bring about an example advantage of estimating a proper stress level while taking into consideration whether a subject is within working hours or outside the working hours when measurement data is measured. Note that the functions of the information processing apparatus 4 can be realized by at least one processor. Therefore, the subjects of the foregoing processes can be read as at least one processor.

### Third Example Embodiment

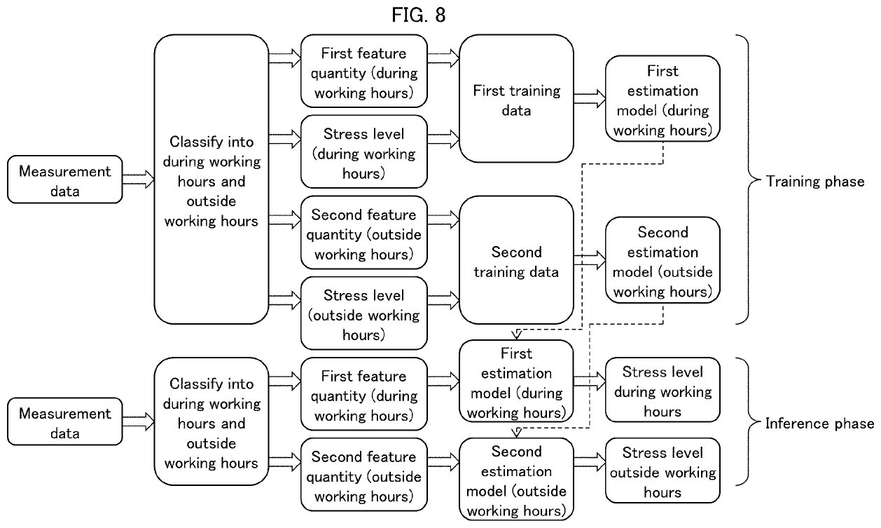

The following description will discuss a third example embodiment of the present invention in detail with reference to the drawings. FIG. 8 is a diagram illustrating overviews of a training data generation method, an estimation model generation method, and a stress level estimation method according to the present example embodiment. A difference from the second example embodiment is that estimation models used are different between a period during working hours and a period outside the working hours. The following description will discuss an example of causing the information processing apparatus 4 illustrated in FIG. 4 to carry out these methods.

In a training phase, similarly to the second example embodiment, the measurement data acquisition section 401 of the information processing apparatus 4 acquires measurement data pertaining to a stress level that indicates a degree of stress of each of one or more subjects, and the classification section 404 classifies the measurement data into first measurement data measured during working hours of the subject and second measurement data measured outside the working hours. Then, the feature quantity calculation section 405 calculates a first feature quantity from the first measurement data, and calculates a second feature quantity from the second measurement data. The processes carried out so far in the training phase are similar to those in the example of FIG. 3.

Here, in the information processing apparatus 4 according to the second example embodiment, the training data generation section 406 generates first training data while associating, as correct answer data, the stress level of the subject during the working hours with the first feature quantity. Moreover, the training data generation section 406 generates second training data while associating, as correct answer data, the stress level of the subject outside the working hours with the second feature quantity. Note that the stress level during working hours and the stress level outside the working hours are calculated by the stress level calculation section 403 based on a result of a questionnaire answered by the subject. The training data generation section 406 may generate training data using other pieces of data in addition to measurement data, similarly to the example of FIG. 3.

In the information processing apparatus 4 according to the second example embodiment, the training process section 407 carries out machine learning using a plurality of pieces of first training data generated as described above. Thus, a first estimation model is generated which is an estimation model for estimating a stress level during working hours, for which the first feature quantity is used as an explanatory variable, and from which a stress level is obtained as an objective variable. Note that, in the generation of the first training data, in a case where other pieces of data are used in addition to measurement data, such other pieces of data are also used as explanatory variables. This also applies to a second estimation model described below.

Moreover, the training process section 407 carries out machine learning using a plurality of pieces of second training data generated as described above. Thus, a second estimation model is generated which is an estimation model for estimating a stress level outside working hours, for which the second feature quantity is used as an explanatory variable, and from which a stress level is obtained as an objective variable.

In an inference phase (stress level determination method), the measurement data acquisition section 401 acquires measurement data which has been measured during a predetermined time period and which pertains to a stress level that indicates a degree of stress of the subject. Note that, in a case where other pieces of data are included in explanatory variables of the first estimation model or the second estimation model, the measurement data acquisition section 401 also acquires such other pieces of data.

Next, the classification section 404 classifies the acquired measurement data into first measurement data measured during working hours of the subject and second measurement data measured outside the working hours. Then, the feature quantity calculation section 405 calculates a first feature quantity from the first measurement data, and calculates a second feature quantity from the second measurement data. The processes carried out so far in the inference phase are similar to those in the example of FIG. 3.

In the information processing apparatus 4 according to the second example embodiment, the inference section 408 estimates a stress level of a subject during working hours using the first estimation model. Specifically, the inference section 408 obtains an estimation value of the stress level of the subject during the working hours by inputting the first feature quantity into the first estimation model. Similarly, the inference section 408 estimates the stress level of the subject outside working hours using the second estimation model. Specifically, the inference section 408 obtains an estimation value of the stress level of the subject outside the working hours by inputting the second feature quantity into the second estimation model.

Note that the inference section 408 may calculate a stress level in the entire predetermined time period including the working hours and the period outside the working hours using the stress level outside the working hours and the stress level during the working hours which have been calculated as described above. For example, the inference section 408 may calculate an arithmetic average value, a weighted average value, or the like of the stress level outside the working hours and the stress level during the working hours as the stress level in the entire predetermined time period.

In the training phase, in a case where the stress level in the entire predetermined time period including the working hours and the period outside the working hours is also known, the training data generation section 406 may generate third training data described in the second example embodiment. That is, the training data generation section 406 may generate third training data while associating the first feature quantity and the second feature quantity with the stress level of the entire predetermined time period.

Note that the feature quantity calculation section 405 does not necessarily need to calculate both the first feature quantity and the second feature quantity, and only needs to calculate at least one of the first feature quantity and the second feature quantity. Then, the training data generation section 406 may generate at least one of first training data and second training data using one of the first feature quantity and the second feature quantity which has been calculated by the feature quantity calculation section 405. In a case where generated training data is one of the first training data and the second training data, an inference model generated by the training process section 407 is also one of the first inference model and the second inference model. The same applies to the inference phase. That is, in a case where one of the first feature quantity and the second feature quantity has been calculated, the inference section 408 estimates at least one of the stress level during working hours and the stress level outside the working hours using one of the first feature quantity and the second feature quantity which has been calculated by the feature quantity calculation section 405.

As described above, the stress level determination method according to the present example embodiment employs the configuration of further including at least one of: calculating, by the information processing apparatus 4, a first feature quantity from the first measurement data; and calculating, by the information processing apparatus 4, a second feature quantity from the second measurement data. Then, in the estimating of the stress level, at least one of estimating of the stress level of the subject during the working hours using a first estimation model and estimating of the stress level of the subject outside the working hours using a second estimation model is carried out. Here, in the first estimation model, the first feature quantity calculated from the first measurement data is used as an explanatory variable, and the stress level is obtained as an objective variable, and in the second estimation model, the second feature quantity calculated from the second measurement data is used as an explanatory variable, and the stress level is obtained as an objective variable. Note that the functions of the information processing apparatus 4 can be realized by at least one processor. Therefore, the subjects of the foregoing processes can be read as at least one processor.

By using the first estimation model for estimating the stress level based on the first measurement data, it is possible to carry out estimation while taking into consideration that measurement data used is first measurement data measured during working hours. Therefore, it is possible to estimate a proper stress level during working hours of the subject.

Moreover, by using the second estimation model for estimating the stress level based on the second measurement data, it is possible to carry out estimation while taking into consideration that measurement data used is second measurement data measured outside working hours. Therefore, it is possible to estimate a proper stress level outside working hours of the subject.

Therefore, according to the stress level determination method of the present example embodiment, it is possible to bring about an example advantage of estimating a proper stress level while taking into consideration whether a subject is within working hours or outside the working hours when measurement data is measured, in addition to the example advantage that is brought about by the stress level determination method according to the first example embodiment.

[Variation]

In the stress level estimation method described in the foregoing example embodiments, the classification section 404 classifies measurement data into two types, i.e., measurement data during working hours and measurement data outside the working hours. However, classification is not limited to these two types. For example, the classification section 404 may classify measurement data outside working hours (i.e., second measurement data) into a plurality of types in accordance with a status of a subject at a time when the second measurement data has been measured. In this case, a total number of classification types is three or more. The estimation section 408 then estimates a stress level based on a result of the classification.

For example, the classification section 404 may classify the second measurement data into “outside working hours away from home” and “outside working hours at home”. In this case, the feature quantity calculation section 405 may use measurement data that is classified into “outside working hours away from home” as measurement data “outside working hours”, and may not use measurement data that is classified into “outside working hours at home”. Thus, the estimation section 408 estimates the stress level without taking into consideration the measurement data “outside working hours at home”.

According to the configuration, it is possible to increase accuracy in estimating the stress level in a case where a relevance is low between measurement data “outside working hours at home” and the stress level of the subject. For example, in a case where a subject does not move so much at home, measurement data “outside working hours at home” is less related to a stress level of the subject, as compared with measurement data “during working hours” and “outside working hours away from home”. Therefore, for such a subject, it is expected to improve accuracy in estimating the stress level by not using measurement data classified as measurement data “outside working hours at home”.

The measurement data “outside working hours at home” can be dealt with as measurement data “during working hours”. In this case, the feature quantity calculation section 405 calculates a first feature quantity using measurement data “during working hours” and measurement data “outside working hours at home”, and calculates a second feature quantity using measurement data “outside working hours away from home”. After that, the estimation section 408 may estimate the stress level of the subject in a manner similar to that in the foregoing second or third example embodiment.

For a subject who performs many obligatory movements such as household work at home, it is considered that measurement data “outside working hours at home” is positively correlated with a stress level. Therefore, for such a subject, by dealing with measurement data “outside working hours at home” as measurement data “during working hours”, it is possible to expect improvement in stress level estimation accuracy.

In a case where there are three or more types of classification, the feature quantity calculation section 405 may calculate different feature quantities for the respective types of classification, as with the case of two types (i.e., during working hours and outside working hours). For example, the feature quantity calculation section 405 may calculate a first feature quantity from measurement data “during working hours”, calculate a second feature quantity from measurement data “outside working hours away from home”, and calculate a third feature quantity from measurement data “outside working hours at home”. In this case, similarly to the foregoing second example embodiment, the estimation section 408 may estimate the stress level using an estimation model 416 for which all of these feature quantities are used as explanatory variables. Alternatively, similarly to the foregoing third example embodiment, the estimation section 408 may estimate the stress level using estimation models 416 that vary for the respective feature quantities.

Note that, in the case of carrying out estimation based on three or more classification results as described above, the training data generation section 406 generates training data 415 using measurement data classified in a manner similar to that at estimation, and the training process section 407 generates an estimation model 416 using that training data 415.

As described above, the stress level estimation method according to the present example embodiment employs the configuration in which the information processing apparatus 4 classifies the second measurement data into a plurality of types in accordance with a status of the subject at a time when the second measurement data has been measured; and the information processing apparatus 4 estimates the stress level of the subject based on a result of the classification.

Therefore, according to the stress level estimation method of this variation, it is possible to bring about an example advantage of carrying out estimation with higher accuracy while taking into consideration a status of a subject outside working hours, in addition to the example advantage that is brought about by the stress level estimation method according to the second example embodiment. Note that the functions of the information processing apparatus 4 can be realized by at least one processor. Therefore, the subjects of the foregoing processes can be read as at least one processor.

[Software Implementation Example]

The functions of part of or all of the information processing apparatuses 1 through 4 can be realized by hardware such as an integrated circuit (IC chip) or can be alternatively realized by software.

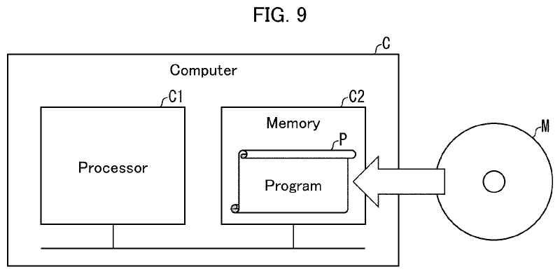

In the latter case, each of the information processing apparatuses 1 through 4 is realized by, for example, a computer that executes instructions of a program that is software realizing the foregoing functions. FIG. 9 illustrates an example of such a computer (hereinafter, referred to as “computer C”). The computer C includes at least one processor C1 and at least one memory C2. The memory C2 stores a program P for causing the computer C to function as the information processing apparatuses 1 through 4. In the computer C, the processor C1 reads the program P from the memory C2 and executes the program P, so that the functions of the information processing apparatuses 1 through 4 are realized.

As the processor C1, for example, it is possible to use a central processing unit (CPU), a graphic processing unit (GPU), a digital signal processor (DSP), a micro processing unit (MPU), a floating point number processing unit (FPU), a physics processing unit (PPU), a microcontroller, or a combination of these. The memory C2 can be, for example, a flash memory, a hard disk drive (HDD), a solid state drive (SSD), or a combination of these.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a non-transitory tangible storage medium M which is readable by the computer C. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communications network, a broadcast wave, or the like. The computer C can obtain the program P also via such a transmission medium.

[Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

[Additional Remark 2]

Some of or all of the foregoing example embodiments can also be described as below. Note, however, that the present invention is not limited to the following supplementary notes.

A method for estimating a stress level according to supplementary note 1 includes: classifying, by at least one processor, measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and estimating, by the at least one processor, a stress level of the subject using at least one of the first measurement data and the second measurement data.

According to the configuration, whether the subject is within working hours or outside the working hours when measurement data is measured is taken into consideration. Therefore, it is possible to estimate a stress level with higher accuracy.

The method according to supplementary note 2 employs, in addition to the configuration of supplementary note 1, a configuration of including: calculating, by the at least one processor, a first feature quantity from the first measurement data; and calculating, by the at least one processor, a second feature quantity from the second measurement data, in the estimating of the stress level, the stress level of the subject being estimated using an estimation model for which the first feature quantity and the second feature quantity are used as explanatory variables, and from which a stress level is obtained as an objective variable.

According to the configuration, a stress level is estimated while using measurement data as a feature quantity that varies depending on whether the measurement data is first measurement data measured during working hours or second measurement data measured outside the working hours. Therefore, according to the configuration, it is possible to estimate a proper stress level while taking into consideration whether a subject is within working hours or outside the working hours when measurement data is measured.

The method according to supplementary note 3 employs, in addition to the configuration of supplementary note 1, a configuration of further including at least one of: calculating, by the at least one processor, a first feature quantity from the first measurement data; and calculating, by the at least one processor, a second feature quantity from the second measurement data, in the estimating of the stress level, at least one of estimating of the stress level of the subject during the working hours using a first estimation model and estimating of the stress level of the subject outside the working hours using a second estimation model being carried out, in the first estimation model, the first feature quantity calculated from the first measurement data being used as an explanatory variable, and a stress level being obtained as an objective variable, and in the second estimation model, the second feature quantity calculated from the second measurement data being used as an explanatory variable, and a stress level being obtained as an objective variable.

According to the configuration, it is possible to estimate a proper stress level while taking into consideration whether a subject is within working hours or outside the working hours when measurement data is measured.

The method according to supplementary note 4 employs, in addition to the configuration of any one of supplementary notes 1 through 3, a configuration in which: the at least one processor classifies the second measurement data into a plurality of types in accordance with a status of the subject at a time when the second measurement data has been measured; and the at least one processor estimates the stress level of the subject based on a result of the classification.

According to the configuration, it is possible to bring about an example advantage of carrying out estimation with higher accuracy while taking into consideration a status of a subject outside working hours.

A method according to supplementary note 5 for generating training data includes: classifying, by at least one processor, measurement data into first measurement data and second measurement data, the measurement data pertaining to a stress level that indicates a degree of stress of each of one or more subjects, the first measurement data having been measured during working hours of the subject, and the second measurement data having been measured outside the working hours; and generating, by the at least one processor, at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

By using the training data generated by the above configuration, it is possible to construct an estimation model that is capable of estimating a stress level while taking into consideration whether a subject is within working hours or outside the working hours when measurement data is measured.

A method according to supplementary note 6 for generating an estimation model includes: acquiring, by at least one processor, at least one of the first training data, the second training data, and the third training data which are described in supplementary note 4; and at least one of (1) generating, by the at least one processor, a first estimation model for which the first feature quantity is used as an explanatory variable, the first estimation model being generated by training using the first training data, (2) generating, by the at least one processor, a second estimation model for which the second feature quantity is used as an explanatory variable, the second estimation model being generated by training using the first training data, and (3) generating, by the at least one processor, a third estimation model for which the first feature quantity and the second feature quantity are used as explanatory variables, the third estimation model being generated by training using the third training data.

According to the configuration, it is possible to construct an estimation model that is capable of estimating a stress level while taking into consideration whether a subject is within working hours or outside the working hours when measurement data is measured.

An information processing apparatus according to supplementary note 7, includes: a classification means that classifies measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and an estimation means that estimates a stress level of the subject using at least one of the first measurement data and the second measurement data.

According to the configuration, whether the subject is within working hours or outside the working hours when measurement data is measured is taken into consideration. Therefore, it is possible to estimate a stress level with higher accuracy.

An information processing apparatus according to supplementary note 8 includes: a classification means that classifies measurement data into first measurement data and second measurement data, the measurement data pertaining to a stress level that indicates a degree of stress of each of one or more subjects, the first measurement data having been measured during working hours of the subject, and the second measurement data having been measured outside the working hours; and a training data generation means that generates at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

By using the training data generated by the above configuration, it is possible to construct an estimation model that is capable of estimating a stress level while taking into consideration whether a subject is within working hours or outside the working hours when measurement data is measured.

A stress level estimation program according to supplementary note 9 is a program for causing a computer to function as an information processing apparatus, the stress level estimation program causing the computer to function as: a classification means that classifies measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and an estimation means that estimates a stress level of the subject using at least one of the first measurement data and the second measurement data.

According to the configuration, whether the subject is within working hours or outside the working hours when measurement data is measured is taken into consideration. Therefore, it is possible to estimate a stress level with higher accuracy.

A training data generation program according to supplementary note 10 is a program for causing a computer to function as an information processing apparatus, the training data generation program causing the computer to function as: a classification means that classifies measurement data into first measurement data and second measurement data, the measurement data pertaining to a stress level that indicates a degree of stress of each of one or more subjects, the first measurement data having been measured during working hours of the subject, and the second measurement data having been measured outside the working hours; and a training data generation means that generates at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

By using the training data generated by the above configuration, it is possible to construct an estimation model that is capable of estimating a stress level while taking into consideration whether a subject is within working hours or outside the working hours when measurement data is measured.

[Additional Remark 3]

Furthermore, some of or all of the foregoing example embodiments can also be expressed as below. Note that each of information processing apparatuses below can further include a memory. In the memory, a program for causing the at least one processor to execute the processes can be stored. The program can be stored in a computer-readable non-transitory tangible storage medium.

An information processing apparatus including at least one processor, the at least one processor carrying out: a process of classifying measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours; and a process of estimating a stress level of the subject using at least one of the first measurement data and the second measurement data.

An information processing apparatus including at least one processor, the at least one processor carrying out: a process of classifying measurement data into first measurement data and second measurement data, the measurement data pertaining to a stress level that indicates a degrees of stress of each of one or more subjects, the first measurement data having been measured during working hours of the subject, and the second measurement data having been measured outside the working hours; and a process of generating at least one of (1) first training data in which the stress level of the subject is associated with a first feature quantity calculated from the first measurement data, (2) second training data in which the stress level of the subject is associated with a second feature quantity calculated from the second measurement data, and (3) third training data in which the stress level of the subject is associated with the first feature quantity and the second feature quantity.

### REFERENCE SIGNS LIST

1: Information processing apparatus
11: Classification section
12: Training data generation section
3: Information processing apparatus
31: Classification section
32: Estimation section
4: Information processing apparatus
404: Classification section
406: Training data generation section
408: Estimation section

What is claimed is:

1. A method for estimating a stress level, comprising:
classifying, by at least one processor, measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours;
estimating, by the at least one processor, a stress level of the subject using the first measurement data and the second measurement data;
calculating, by the at least one processor, a first feature quantity from the first measurement data; and
calculating, by the at least one processor, a second feature quantity from the second measurement data,
in the estimating of the stress level, the stress level of the subject being estimated using an estimation model for which the first feature quantity and the second feature quantity are used as explanatory variables, and from which a stress level is obtained as an objective variable.

2. (canceled)

3. A method for estimating a stress level, comprising:
classifying, by at least one processor, measurement data into first measurement data and second measurement data, the measurement data having been measured during a predetermined time period and pertaining to a stress level that indicates a degree of stress of a subject, the first measurement data having been measured during working hours of the subject, and the second measurement data having been measured outside the working hours;
estimating, by the at least one processor, a stress level of the subject using at least one of the first measurement data and the second measurement data,
said method further comprising at least one of:
calculating, by the at least one processor, a first feature quantity from the first measurement data; and
calculating, by the at least one processor, a second feature quantity from the second measurement data,
in the estimating of the stress level, at least one of estimating of the stress level of the subject during the working hours using a first estimation model and estimating of the stress level of the subject outside the working hours using a second estimation model being carried out,
in the first estimation model, the first feature quantity calculated from the first measurement data being used as an explanatory variable, and a stress level being obtained as an objective variable, and
in the second estimation model, the second feature quantity calculated from the second measurement data being used as an explanatory variable, and a stress level being obtained as an objective variable.

4. The method according to claim 1, wherein:
the at least one processor classifies the second measurement data into a plurality of types in accordance with a status of the subject at a time when the second measurement data has been measured; and
the at least one processor estimates the stress level of the subject based on a result of the classification.

5. A method for generating training data, said method comprising:
classifying, by at least one processor, measurement data into first measurement data and second measurement data, the measurement data pertaining to a stress level that indicates a degree of stress of each of one or more subjects, the first measurement data having been measured during working hours of the subject, and the second measurement data having been measured outside the working hours; and
generating, by the at least one processor, at least one of
(1) first training data in which the stress level of the subject is associated with a first feature quantity calculated from the first measurement data,
(2) second training data in which the stress level of the subject is associated with a second feature quantity calculated from the second measurement data, and
(3) third training data in which the stress level of the subject is associated with the first feature quantity and the second feature quantity.

6. A method for generating an estimation model, said method comprising:
acquiring, by at least one processor, at least one of the first training data, the second training data, and the third training data which have been generated by the method recited in claim 5; and
at least one of
(1) generating, by the at least one processor, a first estimation model for which the first feature quantity is used as an explanatory variable, the first estimation model being generated by training using the first training data,
(2) generating, by the at least one processor, a second estimation model for which the second feature quantity is used as an explanatory variable, the second estimation model being generated by training using the second training data, and
(3) generating, by the at least one processor, a third estimation model for which the first feature quantity and the second feature quantity are used as explanatory variables, the third estimation model being generated by training using the third training data.

7-8. (canceled)

9. A computer-readable non-transitory storage medium storing a stress level estimation program for causing a computer to carry out the classifying of measurement data, the calculating of a first feature quantity, the calculating of a second feature quantity, and the estimating of a stress level which are recited in claim 1.

10. A computer-readable non-transitory storage medium storing a training data generation program for causing a computer to carry out the classifying of measurement data and the generating of at least one of first training data, second training data, and third training data which are recited in claim 5.

11. A computer-readable non-transitory storage medium storing a stress level estimation program for causing a computer to carry out the classifying of measurement data and the estimating of a stress level which are recited in claim 3, the stress level estimation program causing the computer to further carry out at least one of the calculating of a first feature quantity and the calculating of a second feature quantity which are recited in claim 3.
